(19) United States
(12) Patent Application Publication
(10) Pub. No.: US 2024/0374192 A1
(43) Pub. Date: Nov. 14, 2024

(54) SYSTEMS AND METHODS FOR QUANTIFYING HYPERTONUS

(71) Applicant: Rehabilitation Institute of Chicago d/b/a Shirley Ryan AbilityLab, Chicago, IL (US)

(72) Inventors: William Zev Rymer, Chicago, IL (US); Alexander Barry, Chicago, IL (US); Leah O'Shea, Chicago, IL (US); Arthur Prochazka, Chicago, IL (US)

(21) Appl. No.: 18/658,887

(22) Filed: May 8, 2024

Related U.S. Application Data
(60) Provisional application No. 63/464,919, filed on May 8, 2023.

(51) Int. Cl. A61B 5/22; A61B 5/11

(52) U.S. Cl. CPC A61B 5/224; A61B 5/1121; A61B 2562/0219; A61B 2562/0247; A61B 2562/04; A61B 2562/06

(57) ABSTRACT

A device for improved quantification of hypertonus is disclosed. The device is configured for grasping a limb segment of a patient and includes a force sensor module for continuously measuring a force generated by rotating the limb segment of the patient between a first position and a second position, at least one IMU, and at least one processor that generates at least one clinically relevant measure of response based at least on force data from the force sensor module and inertial data from the IMU.

### CROSS REFERENCE TO RELATED APPLICATIONS

This is a non-provisional patent application that claims benefit to U.S. Provisional Patent Application Ser. No. 63/464,919 filed on May 8, 2023, which is herein incorporated by reference in its entirety.

### FIELD

The present disclosure generally relates to an apparatus and related methods for quantifying hypertonus in a body segment of a person with a sensorimotor disorder and more specifically to improved apparatus and method for measuring velocity-dependent resistance of a body segment to externally imposed motion.

### BACKGROUND

The field of medicine is filled with precision measurements for diseases, conditions, and injuries. In rehabilitation and physical medicine, however, comprehensive evaluations are extremely limited as most evaluations are based on subjective assessments by trained clinicians as opposed to true biomarkers. Following neurologic injuries (especially, stroke, cerebral palsy, spinal cord injury, amyotrophic lateral sclerosis, traumatic brain injury, multiple sclerosis, muscular dystrophy, Parkinson's Disease), there is often an increase in the resistance of a limb to movement (tone, or tonus). An abnormal increase in stiffness is called hypertonus. This can limit activities of daily living, leading to a decrease in quality of life. A common form of hypertonus is spasticity, which is a feature of upper motor neuron syndrome (UMNS) that is defined by a velocity-dependent increase in muscle tone due to hyperexcitability of the stretch reflex. In clinical practice, tonus is described as being either normal, flaccid, hypotonic, or hypertonic. Within the hypertonic domain, clinicians rate stiffness levels from 0 to 4.

Clinicians may rate the degree of spastic hypertonus by assessing a limb's level of mechanical resistance to imposed movement, in accordance with the Ashworth Scale (AS) or the Modified Ashworth Scale (MAS). The Modified Ashworth Scale (MAS) involves quickly rotating a limb segment about a joint and assessing a “catch” point, the “catch” being a sudden increase in resistance to the imposed movement. Although these scales are commonly used, their use as an assessment of spastic hypertonus has been repeatedly criticized due to inaccuracy, having poor granularity, ambiguity in wording, lack of standardization, poor intra-and inter-rater reliability in post-stroke populations, and only being a measure of resistance to externally-imposed or passive motion. An alternative to the AS and MAS is the Tardieu Scale (TS), which is similar to the MAS, but also takes into account slow movements. The TS has been cited as a more valid means of assessing the neural component of spasticity due to addressing the velocity-dependent component and having adequate intra-and inter-rater reliability. Even so, the reliability and validity of the TS is still questioned and publications assessing reliability and validity, particularly in the adult population, are scarce and inconclusive. To capture the response of a limb to rotation of limb segments about joints, clinicians typically use a goniometer which, while accurate if used correctly, adds time to the evaluation.

Others have proposed measuring devices to improve quantification of spasticity, including devices that incorporate EMG sensor data, various imaging techniques, or linear actuators, or systems for moving limbs at specified constant velocities. However, these advanced devices are often highly resource intensive, have a cumbersome design, and are limited to be used for a specific joint. These known devices involve complex instrumentation and large peripherals, which take time to set up and may be challenging to manage in a clinical environment, resulting in poor adoption due to these constraints. In the clinic, physicians and therapists can and need to be able to complete a battery of assessments within a few minutes. Therefore, none of these known devices have been consistently adopted in the clinical environment.

In one example, a device was proposed that measured the force applied to a limb by computing the difference in pressures sensed within a pair of inflated pads held on either side of the limb. In practice, the inflated pads were bulky and made it difficult for an assessor to grasp a limb. Another device was proposed in which a pair of force sensors, described as “sandwich plates” were held in an adjustable, rigid, U-shaped bracket, which was attached to a patient's limb and measured applied torques. The device as described is bulky and difficult for an assessor to use. Further, these devices are not capable of identifying a catch point, if present. The angle of catch is a clinically important continuous measure of spasticity, i.e. the earlier into a patient's range of motion a catch occurs, the more severe the spasticity as it is a velocity-dependent stretch response which can lead to an abrupt change before the end of the range of motion. This measure can dictate the course of therapy and interventions. For example, in serial casting, the spastic limb will be casted at a greater angle than the catch as a means to passively stretch the muscle over time and increase the catch angle towards full extension.

Thus, a need exists for a novel, robust, low-profile and simple device and method of spastic hypertonus assessment for clinician use. Specifically, a device is needed that can accurately measure applied force, joint angular velocity, and joint acceleration to assess the stiffness of a spastic limb, while remaining fast and easy to use.

### SUMMARY

The present disclosure relates to a simplified summary of various aspects described herein. This summary is not an extensive overview, and is not intended to identify key or critical elements or to delineate the scope of the claims. The following summary merely presents some concepts in a simplified form as an introductory prelude to the more detailed description provided below. Corresponding apparatus, systems, and computer-readable media are also within the scope of the disclosure.

The MITSS (Modified InTelligent Scale for Spasticity) device described herein presents a novel method of hypertonus assessment for clinician use through the implementation of a wearable device that utilizes force-sensing plates coupled with an inertial measurement unit (IMU) sensor to collect real-time force, position, acceleration, and velocity data during passive movement.

In one aspect, the system may include a device for grasping a body segment of a patient, such as a mitt, cuff, or glove. An IMU that measures acceleration and position may be incorporated directly into the grasping device, along with force sensors. With these features, the system may provide quantitative information on the force and movement (position, velocity, acceleration) applied to the limb. This information may be used to derive measures of hypertonus, such as catch positions, clonus (tremor) magnitude and period, and cogwheel behavior. A secondary IMU, positioned on another body segment for example, can be incorporated into the system to increase specificity by finding absolute differences in orientations. The system may further include a processor with instructions in the form of an algorithm that, when executed, cause the processor to correlate joint response to clinical scales (e.g., MAS, TS). The algorithm computes both the linear acceleration and the resultant force applied to the limb to provide real time measurement of hypertonus. Bluetooth capabilities or other such wireless protocols may be incorporated into the system for up-loading data to other devices.

The system as described herein may be used in a matter of seconds, completely stand-alone from external devices (e.g., external computing devices), to accurately measure elbow angle, acceleration, and applied force and provide an effective means of quantifying spastic hypertonus. The system may be used on any joint to assess hypertonus and may be applicable to additional populations following neurologic injuries, including cerebral palsy, spinal cord injury, Parkinson's Disease, amyotrophic lateral sclerosis, traumatic brain injury, multiple sclerosis, and muscular dystrophy.

These features, along with many others, are discussed in greater detail below. It is with these observations in mind, among others, that various aspects of the present disclosure were conceived and developed.

### BRIEF DESCRIPTION OF THE DRAWINGS

The present disclosure is described by way of example and not limited in the accompanying figures in which like reference numerals indicate similar elements and in which:

FIG. 1 shows a top perspective view of an example of a grasping device according to one or more aspects of the disclosure;

FIG. 2 shows an example of a system for quantifying hypertonus according to one or more aspects of the disclosure;

FIG. 3 shows a top view of an example of a grasping device according to one or more aspects of the disclosure;

FIG. 4 shows a side cross-sectional view of the example grasping device of FIG. 1, according to one or more aspects of the disclosure;

FIG. 5 shows an exploded view of an example of a system for quantifying hypertonus according to one or more aspects of the disclosure;

FIG. 6 shows a top perspective view of an example of a data processing module according to one or more aspects of the disclosure;

FIG. 7 shows an exploded view of an example of a data processing module according to one or more aspects of the disclosure;

FIG. 8 shows a top view of an example circuit board of a data processing module according to one or more aspects of the disclosure;

FIG. 9 shows a top view of an example user interface of a data processing module according to one or more aspects of the disclosure;

FIG. 10 shows a perspective view of an example force sensor module according to one or more aspects of the disclosure;

FIG. 11 shows an exploded view of an example of force sensing module according to one or more aspects of the disclosure;

FIG. 12 shows an exploded view of an example circuit board of a force sensing module according to one or more aspects of the disclosure;

FIG. 13 shows a top view of an example circuit board and sensor plat of a force sensing module according to one or more aspects of the disclosure;

FIG. 14 shows a side view of an example of force sensing module according to one or more aspects of the disclosure;

FIG. 15 shows an example illustration of a signal flow diagram between components according to one or more aspects of the disclosure;

FIG. 16 shows an example system for quantifying hypertonus according to one or more aspects of the disclosure;

FIG. 17 shows an example system for quantifying hypertonus in use according to one or more aspects of the disclosure;

FIG. 18 shows an example system for quantifying hypertonus in use according to one or more aspects of the disclosure;

FIG. 19 shows a flow chart of an example method for quantifying hypertonus according to one or more aspects of the disclosure;

FIG. 20 shows a flow chart of an example method for quantifying hypertonus according to one or more aspects of the disclosure;

FIG. 21 shows a plot of limb position and acceleration data from slow and fast movement trials collected using an example of the system for quantifying hypertonus according to one or more aspects of the disclosure;

FIGS. 22A-22B shows a plot of limb position and acceleration data from paretic and nonparetic limbs collected using an example of the system for quantifying hypertonus according to one or more aspects of the disclosure;

FIGS. 23A-23D shows a plot of limb position and torque data collected using an example of the system for quantifying hypertonus according to one or more aspects of the disclosure;

Corresponding reference characters indicate corresponding elements among the view of the drawings. The headings used in the figures do not limit the scope of the claims.

### DETAILED DESCRIPTION

In the following description, reference is made to the accompanying drawings, which form a part hereof, and in which is shown by way of illustration various embodiments in which aspects of the disclosure can be practiced. It is to be understood that other embodiments can be utilized, and structural and functional modifications can be made without departing from the scope of the present disclosure. Aspects of the disclosure are capable of other embodiments and of being practiced or being carried out in various ways. In addition, it is to be understood that the phraseology and terminology used herein are for the purpose of description and should not be regarded as limiting. Rather, the phrases and terms used herein are to be given their broadest interpretation and meaning.

Aspects of the present disclosure relate to novel methods and systems to quantify hypertonus. The inventive methods and systems can be used in a clinical setting to determine the severity of hypertonus as a guide for therapeutic strategies such as medication, splinting and botulinum toxin injections. It can also be used in a home setting by a user with spasticity to track the improvement of therapy.

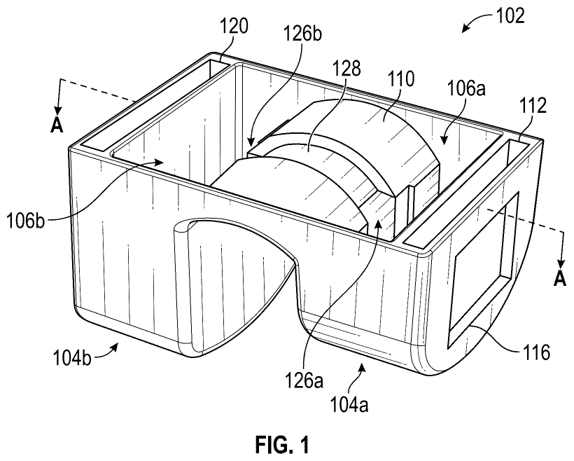

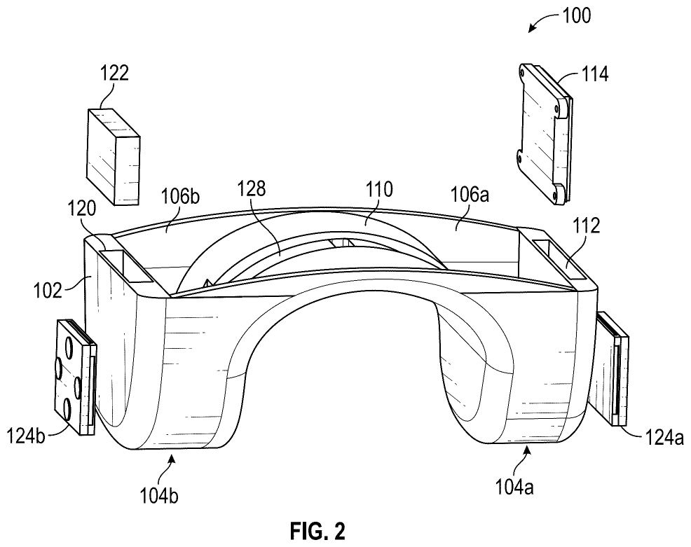

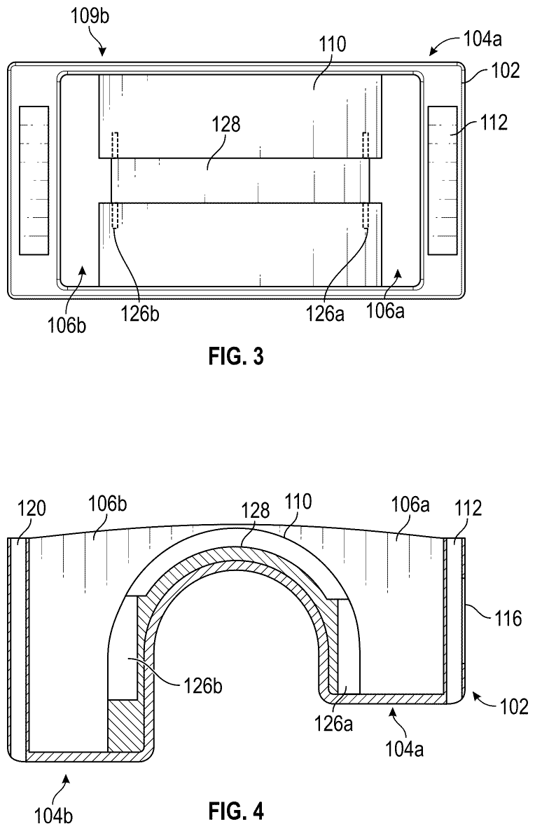

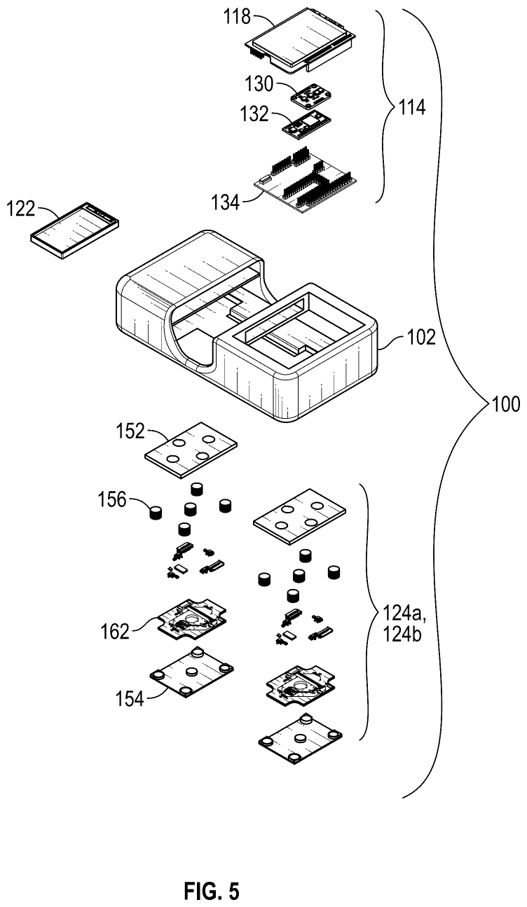

As shown in FIGS. 1-4, the system 100 may include a hand-held grasping device 102, such as a mitt, cuff, or glove. A clinician or other individual may use the device 102 to grasp a limb segment of a patient, such as a forearm, between a thumb segment 104a and a finger segment 104b of the device 102 and move the limb segment through its full range of motion to evaluate spastic hypertonus. Finger segment 104b may be deeper than thumb segment 104a to accommodate typical hand shape. Each segment 104a, 104b defines a cavity 106a, 106b for accepting the thumb and fingers, respectively, of a user's hand and a grip surface 108a, 108b. The thenar space of the user's hand (between the thumb and forefinger) rests on a bridge 110 connecting the two segments 104a, 104b of grasping device 102. In one embodiment, the grasping device 102 is molded in a generally “U” shape, as shown in FIG. 1. Alternatively, the device 102 may be molded generally flat (at shown in FIG. 5) and capable of being folded into a “U” shape during use. The device 102 may be formed of any material or a combination of materials suitable for use in a medical, therapy or rehabilitation environment. Generally, the material(s) of the device should be durable, capable of being sanitized after each use, and relatively lightweight. Further, the material(s), at least of the bridge 110 portion, should be comfortable and flexible enough to allow the user to grasp and apply pressure to a limb segment of a patient. In one example, the grasping device 102 is formed of molded silicone.

The system 100 further includes, in one example, a slot 112 on the thumb segment 104a for accepting a data processing module 114 and a window 116, through which a user interface 118 is accessible. User interface 118 is provided on the thumb segment 104a so that a user of the system 100 may easily view and interact with the user interface 114 while grasping a limb segment of a patient. A slot 120 on the finger segment 104b of the device 102 is provided for accepting a power supply 122. Power supply 122 may be provided as any appropriate source of power, including a battery and other related components. For example, power supply 122 may include a 3.7 V, 120 mAh rechargeable battery and a lithium polymer (LiPo) charger and booster for boosting the voltage of the battery to a constant 5V1A output, suitable for powering the force sensor modules 124a, 124b and data processing module 114. A force sensor module 124a, 124b is positioned adjacent to each of the two grip surfaces 108a, 108b of the grasping device 102 for determining the forces applied to the limb about a joint under evaluation, for example, an elbow, knee, or wrist joint. Each of the force sensor modules 124a, 124b may be releasably inserted into a respective recess 126a, 126b within the device 102. Recesses 114a, 114b may be connected by a channel 128 in bridge 110 for receiving wires or cables. FIG. 5 illustrates an exploded view of the system 100 including grasping device 100, data processing module 114, power supply 122, and force sensor modules 124a, 124b.

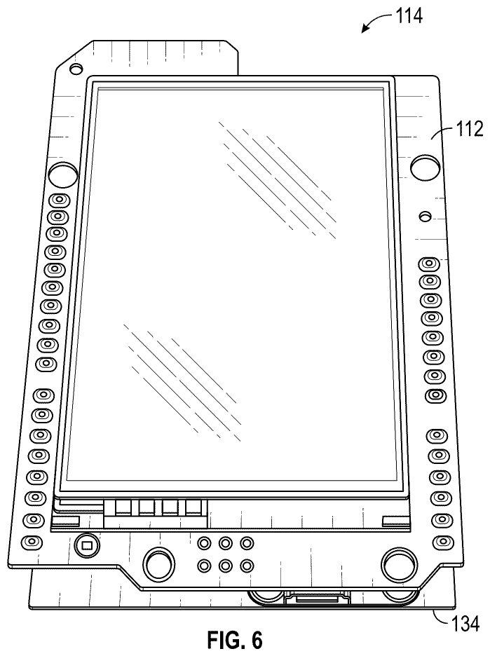

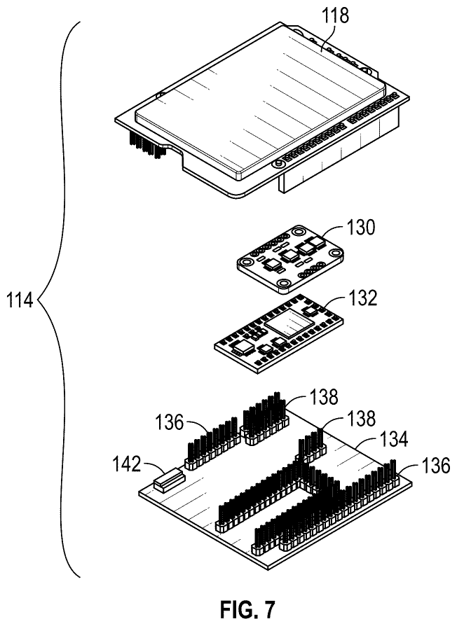

Turning to FIGS. 6-8, data processing module 114 includes at least one inertial measurement unit (IMU) 130 to obtain values to score hypertonus, a microcontroller 132, a circuit board 134, and a user interface 118. The data processing module 114 integrates and controls all the electronic components of the system 100, including the at least one IMU 130, force sensor modules 124a, 124b, the power supply 122, and the user interface 118. In some embodiments, data processing module 114 may be housed in a case (not illustrated). The IMU 130 may be provided as any appropriate device for obtaining inertial data, including an accelerometer, gyroscope and magnetometer. In one example, IMU 130 is a BNO055 Adafruit 9-degrees of freedom absolute orientation device. In some embodiments, system 100 may include more than one IMU. For example, one IMU may be positioned on the grasping device 102 itself. A second IMU may, for additional accuracy, be positioned on a stationary portion of the patient's body or limb under evaluation. For example, if a patient's arm is being evaluated, a clinician will grasp and move the forearm with the grasping device, while the upper arm of the patient is held stationary. Position, acceleration, and gyroscope data from the IMU 130 may be saved to an on-board secure digital (SD) card and transmitted to a microcontroller 132 for filtering and executing mathematical calculations. In one example, microcontroller 132 is provided as a Teensy® 3.2 Microcontroller. Any appropriate microcontroller may be used. Outputs from the microcontroller 132 are sent to the user interface 118 which, in one example, may be a 4Duino-24 screen. Each of the IMU 130, microcontroller 132 and user interface 118 components may communicate with the I2C protocol. Alternatively (or in combination), the position, acceleration, and gyroscope data values from the IMU 130 may be transmitted to an external processor, either by wireless, Bluetooth, or other protocol, and outputs may be displayed on an external display. Data processing module 114 may also include a means for providing auditory, visible, or haptic cues to a user of the system 100 to, for example, provide time cues for initiation and cessation of movement trials. In one example, the data processing module 114 includes a piezoelectric buzzer.

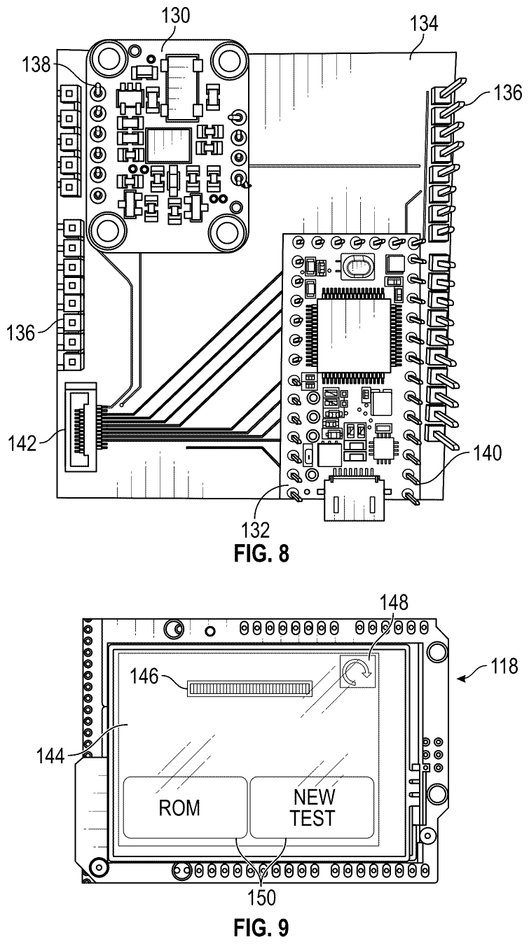

Circuit board 134 includes pins 136 for connecting the user interface 118, pins 138 for connecting the IMU 130, pins 140 for connecting the microcontroller 132 and an input connector 142 for receiving power from the power supply 122 and signals from each of the force plate modules 124a, 124b. Connector 142 may be provided as a flat flexible cable connector. FIG. 8 illustrates the circuit board 134 with the IMU 130 connected at pins 138 and the microcontroller 132 connected at pins 140. As shown in FIG. 6, circuit board 134 may be connected at the back of the user interface 118. Data processing unit 114 may, in some examples, be provided in a housing or case.

User interface 118 may include a display 144, as shown in FIG. 9. Display 144 may be provided as a touchscreen display for conveying information to the user and providing means for the user to interact with the device. For example, the user interface 118 may include a battery charge indicator 146 for representing the amount of power remaining on the power supply 122, a display orientation selector 148 for rotating graphics on the display 144, and one or more trial selectors 150 for starting a new movement trial. Movement trials may include a range of motion (ROM) trial in which the limb is moved at a slow joint velocity, e.g. less than 10 degree/second (“slow trial”), or a trial in which the limb is moved at a fast joint velocity (e.g. up to 180 degree/second) to elicit a catch (“fast trial”). The user interface 118 may also include its own internal processor. Scores or results of tests conducted using system 100 based on data collected by one or more of the IMU 130 and force sensor modules 124a, 124b, and processed on the microcontroller 132, may also be presented on the display 144.

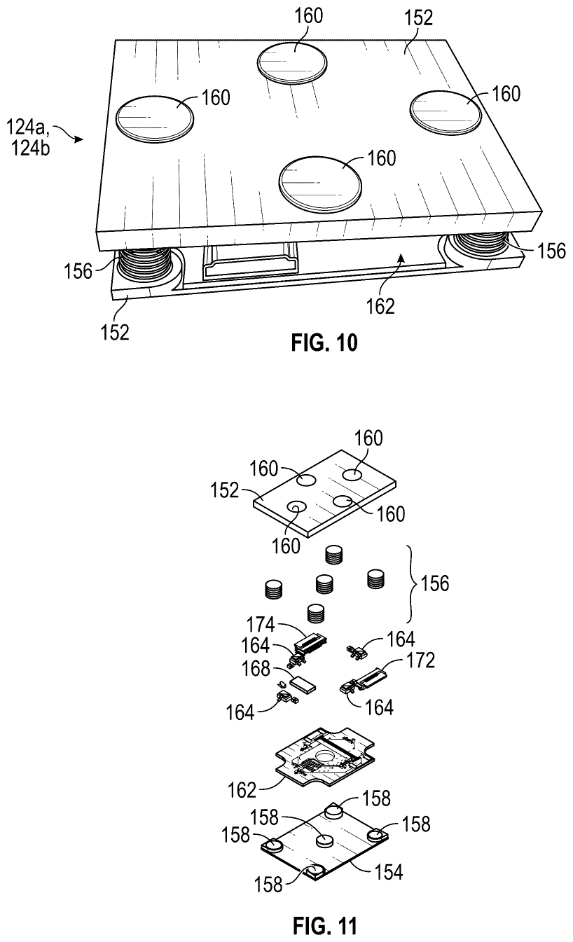

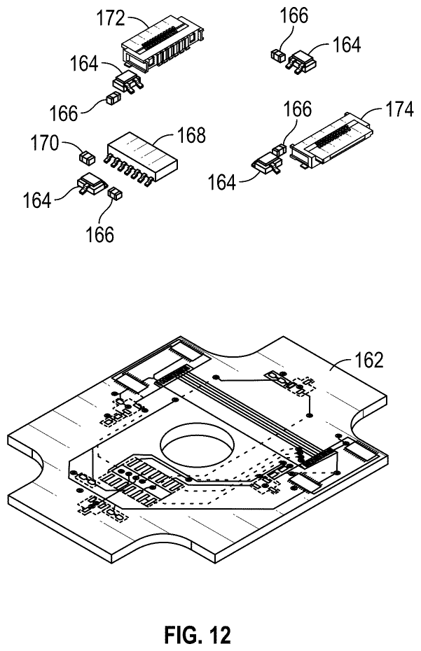

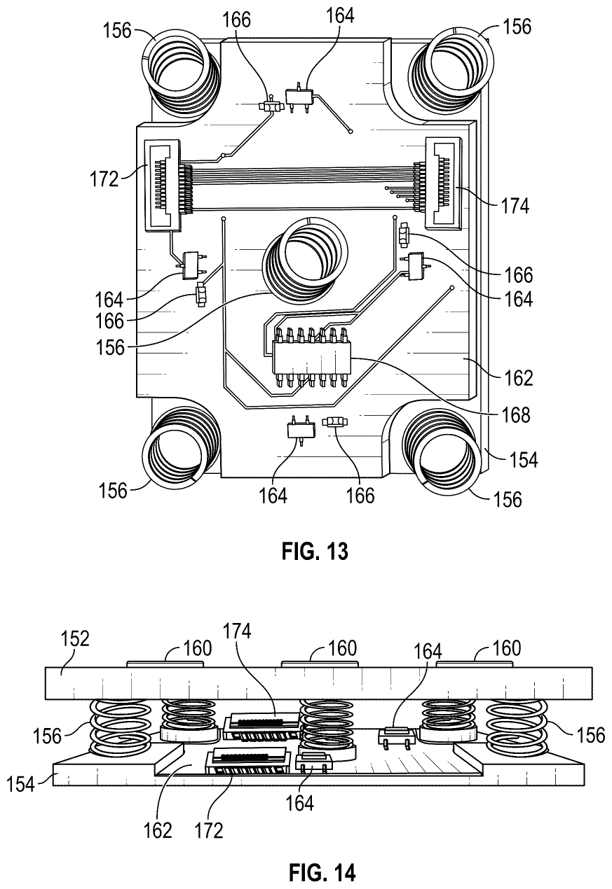

As shown in FIGS. 10-14, in one example, each force sensor module 124a, 124b comprises a pair of opposed plates 152, 154 held apart by one or more springs 156, one or more magnets 160, and a circuit board 162. The opposed plates 152, 154 may be arranged substantially parallel to one another and in relatively close proximity. Any suitable material, including metal, with a very high bending stiffness may be used for the plates 152, 154. In one embodiment, the force sensor module 124a, 124b includes five (5) springs 156, one positioned in proximity to each corner of the plates and one positioned at the center. This positioning compensates for uneven distribution of forces, thereby allowing a user to apply force to any portion of the force sensor module 124a, 124b, while maintaining stability and functionality of the sensors. The springs 156 are mounted to the bottom plate 154, for example, on posts 158. To keep the profile of the force sensor modules 124a, 124b low, the springs 156 may be relatively short and stiff. In one example, the springs 156 are wave disc springs having a spring constant of 66.67 lbs/in. While the terms “top” and “bottom” are used herein to identify each of the plates 152, 154, respectively, the orientation of the force sensor modules 124a, 124b within the grasping device 102 is not important to functioning of the sensors.

The one or more magnets 160 are mounted to the top plate 152 of each module 124a, 124b and positioned to be in alignment with a corresponding Hall Effect sensor 164 on the circuit board 162. In the embodiments shown in the Figures, the magnets 160 are press-fit into openings in the top plate 152, but other means of mounting or affixing the magnets 160 to the top plate 152 are contemplated. In some embodiments, the magnets 160 are oriented with the same polarity. Circuit board 162 is positioned adjacent to the bottom plate 154 so that the springs 156 maintain a space between the Hall Effect sensors 164 and the magnets on the top plate 152. Each Hall Effect sensor 164 may be in electrical communication with a capacitor 166 for connecting the sensor voltage to ground. An operational amplifier (op-amp) 168, and associated capacitor 170 may be provided for amplifying the output of each of the Hall Effect sensors 164. The circuit board 162 further includes an input connector 172 and an output connector 174. The output of the op-amp 168 is in electrical communication with the output connector 174.

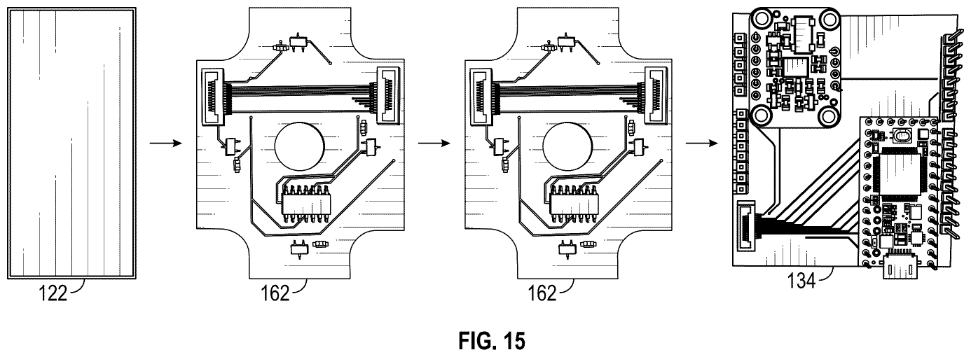

FIG. 15 illustrates the flow of signals from the various systems. Voltage from the power supply 122 is output to a circuit board 162 of the first force sensor module 124a via the input connector 172. Power and Hall Effect sensor signals are output from the first force sensor module 142a via the output connector 174 to the input connector 171 of the circuit board 162 of the second force sensor module 124b. Power and Hall Effect sensor signals from both the first force sensor module 142a the second force sensor module 124b are output via the output connector 174 of the second force sensor module 124b to the circuit board 134 of the data processing module 114 via the input connector 142. Connections between each of the systems as described here may be by ribbon cable.

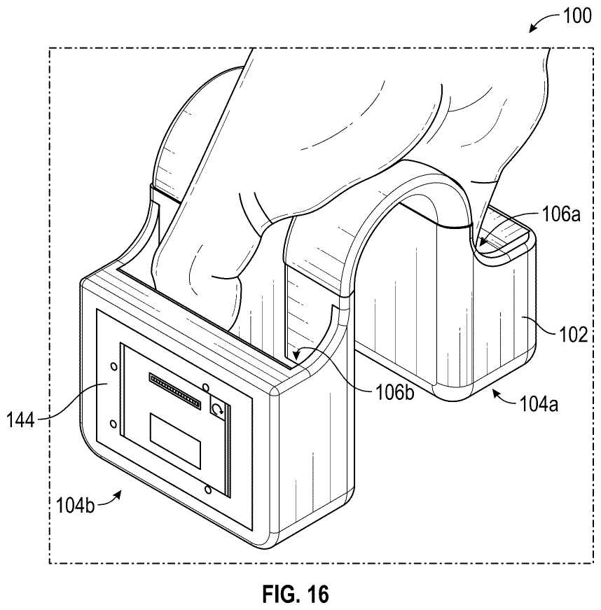

In operation, a user holds the grasping device 102 with their thumb in the cavity 106a of the first segment 104a and their fingers in the cavity 106b of the second segment 104b, as shown in FIG. 16. The user places the grasping device 102 around the limb segment of a patient such that each of grip surfaces 108a, 108b and force sensor modules 124a, 124b are opposing one another and positioned on either side of the grasped limb segment. When a force is applied, squeezing the plates 152, 154 together, the springs 156 shorten, bringing the magnets 160 closer to the Hall Effect sensors 164, whose output signals thereby change in proportion to the force applied. The net (resultant) force applied to the limb is computed from the difference between the output signals from the force sensor modules on opposite sides of the limb. To further clarify, this computation subtracts the compressive force applied by the clinician to the limb segment, leaving the net (resultant force) that moves the limb against the resistance of the muscles. This information may be used to determine force responses over the range of motion which, along with the concurrent movement signals, may be used to determine the angle of catch of the joint under evaluation. Data collected from the IMU allows the system to determine where the moving limb segment is in space and to quantify both the range of motion and the catch angle, with respect to the starting position. Providing a clinician with a catch angle allows quantification of the MAS and TS scales and can improve intra-and inter-rater reliability.

Measurements of spasticity are highly relevant in rehabilitation medicine for quantifying injury severity and determining treatment options. A defining feature of spasticity is an abnormally high resistance to imposed movement. The limb behaves like a spring, so the resistive force increases with increasing amplitude of movement. The resistive force also increases with increasing speed of movement, i.e., in addition to spring stiffness, there is viscous stiffness. Finally, there is often a clear angular threshold for the onset of the reflex response, typically perceived as a “catch” or a transient increase in resistance force.

Presently, the main scales used to determine spastic hypertonus and develop therapy treatment in persons with strokes include the Ashworth Scale (AS), the Modified Ashworth Scale (MAS) and the Tardieu Scale (TS). The AS tests resistance to passive movement about a joint without specifying low or high velocities. Scores range from 0-4, with 5 choices: 0 (0)—No increase in tone; 1 (1)—Slight increase in tone giving a catch when the limb was moved in flexion or extension; 2 (2)—More marked increase in tone but limb easily flexed; 3 (3)—Considerable increase in tone-passive movement difficult; and 4 (4)—Limb rigid in flexion or extension. A score of 1 indicates no hypertonus and 5 indicates a very high level of hypertonus.

The MAS is a revised version of the AS. As compared to the AS, MAS adds a 1+ scoring category to indicate resistance through less than half of the movement. Scores range from 0-4, with 6 choices: 0 (0)—No increase in muscle tone; 1 (1)—Slight increase in muscle tone, manifested by a catch and release or by minimal resistance at the end of the range of motion when the affected part(s) is moved in flexion or extension; 1+ (2)—Slight increase in muscle tone, manifested by a catch, followed by minimal resistance throughout the remainder (less than half) of the range of movement (ROM); 2 (3)—More marked increase in muscle tone through most of the ROM, but affected part(s) is easily moved; 3 (4)—Considerable increase in muscle tone passive, movement is difficult; 4 (5)—Affected part(s) is rigid in flexion or extension. The above grades are assigned by moving a joint/muscle through a high velocity stretch. A feature of the resistive response that contributes to the rating is “a catch,” a sudden increase in resistance, and an abrupt change in angular velocity due to the onset of a stretch reflex.

The TS is a scale for measuring spasticity that is similar to the MAS in that it takes into account resistance to passive movement, but it does so at both slow and fast velocities. The most recent versions of the scale involve two measurements: 1) Quality of muscle reaction; and 2) Angle of muscle reaction. The Quality of Muscle Reaction is scored 0-4: Grade 0: No resistance throughout the course of the passive movement; Grade 1: Slight resistance throughout the course of the passive movement, followed by release; Grade 2: Clear catch at a precise angle, interrupting the passive movement, followed by release; Grade 3: Fatigable clonus (<10 seconds when maintaining pressure) occurring at a precise angle; Grade 4: Non-fatigable clonus (>10 seconds when maintaining pressure) occurring at a precise angle.

The Modified TS describes R1 and R2; R1 is the angle of muscle reaction, R2 is the full ROM. The angle of full ROM (R2) is taken at a very slow speed (V1). The angle of muscle reaction (R1) is defined as the angle at which a catch or clonus is found during a quick stretch (V3). R1 is then subtracted from R2, and this represents the active component of the muscle reflex response.

These clinical assessments have been predominantly used in daily assessments of spasticity in clinics and have been the primary outcome measurements reported in most clinical trials examining drug or rehabilitation treatments for spasticity. The problem with these scales is that they have little granularity, and both have been found to have poor reliability among clinicians. Often, these measurements are affected by the inter-and intra-rater variability, resulting in a reliability issue with the outcome measurement scoring. Moreover, the granularity of the current clinical scales is often a barrier to accurately assessing and tracking the disease progression or recovery and the effects of clinical interventions in patients. Currently, clinical tests, such as the MAS, can discriminate just three or four levels of spasticity. The novel system 100 and method 200 described herein allows clinicians to categorize spasticity more precisely, with at least six to eight levels of severity. The increased measurement resolution provided by the system 100 allows tracking of patient injury courses as well as informing more patient-centered interventions.

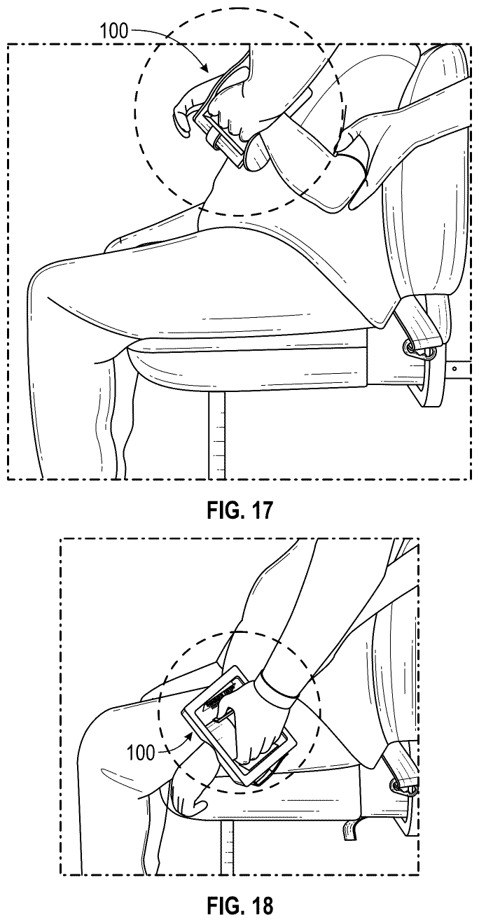

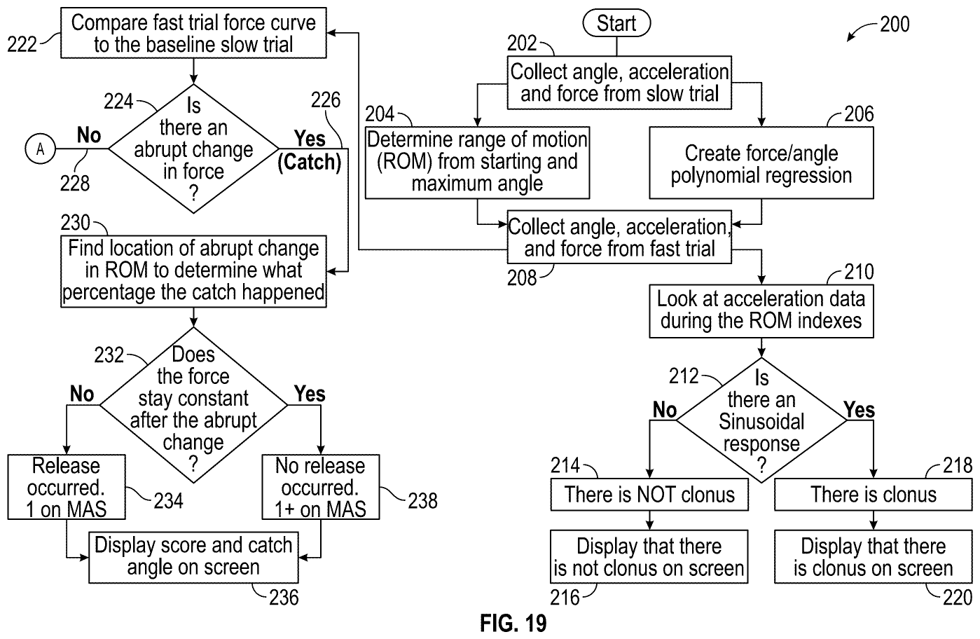

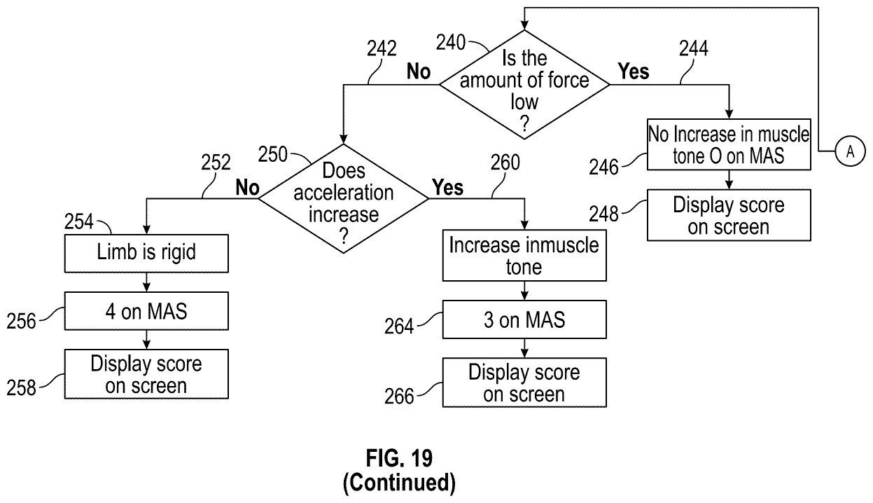

In assessing a patient using the system 100 described herein, the “ROM” or “slow trial” is the first test performed. This test is performed to calibrate the displacement sensing system and create a baseline for each subject. An auditory stimulation may be presented to standardize testing to create a unified speed between clinicians. For example, the system 100 may buzz every time the clinician should position the limb in a fully flexed or fully extended position. For the ROM trial, the clinician places the participant's forearm in a fully flexed position, as shown in FIG. 17. Once the ROM button is pushed, an auditory stimulation (or other notification) will be provided and the user interface 118 tells the microcontroller 132 to start collecting for the ROM trial. The clinician will then extend the forearm, which should be fully extended by the next buzz (˜3 seconds), as shown in FIG. 18. The clinician will then return the forearm to the fully flexed position by the last buzz (˜3 seconds). The clinician and participant will remain in this position until the device saves the data (for example to the micro-SD) and prompts the user to return to the main menu. Next, the clinician performs the “fast trial” also referred to herein as the “New Test” trial. The clinician repeats the same joint movements at a faster velocity indicated by the auditory stimulation for the “fast trial”. Saved data from the ROM trial is called again for computation after the fast trial” is performed. The microcontroller 132 compares the two files to see if any changes occurred in the “fast trial” compared to the “slow trial”. The microprocessor's software algorithm may then provide a computed spasticity score expressed in terms of recognized clinical tests in accordance with the methods described herein.

The present system 100 may also be used to identify the presence of a catch and the catch angle based on acceleration and position data generated by the IMU. Clinically, the catch angle is the joint angle where quick passive muscle stretching induces an abrupt rise in muscle tension. The system 100 characterizes a catch as a sudden change in acceleration or shift in position data. In participants with a catch, or who have increased resistance during the “fast trial,” the reaction forces from the clinician's hand to the force sensor assemblies 124a, 124b capture these changes. As the forearm is moved into extension, a catch in the elbow flexor muscles will cause the elbow to suddenly and involuntarily flex, causing a larger responsive applied-force by the thumb of the clinician and resulting in an increase in the force sensed by the thumb-side force sensor module 124b, and a slowing in movement. The opposite happens when the forearm moves from a fully extended position to a fully flexed position. Here, when the forearm is moved into flexion, reflex activation of the elbow extensor muscles cause the elbow to involuntarily extend, increasing the reaction force by the clinician's fingers, thereby increasing the force sensed by the finger-side force sensor module 124a, and slowing the flexion movement.

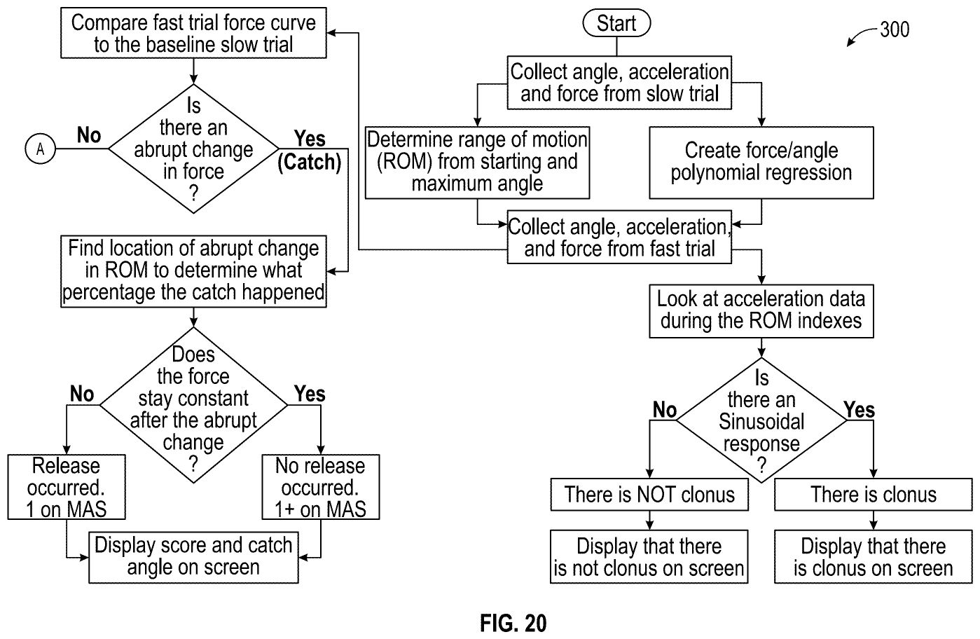

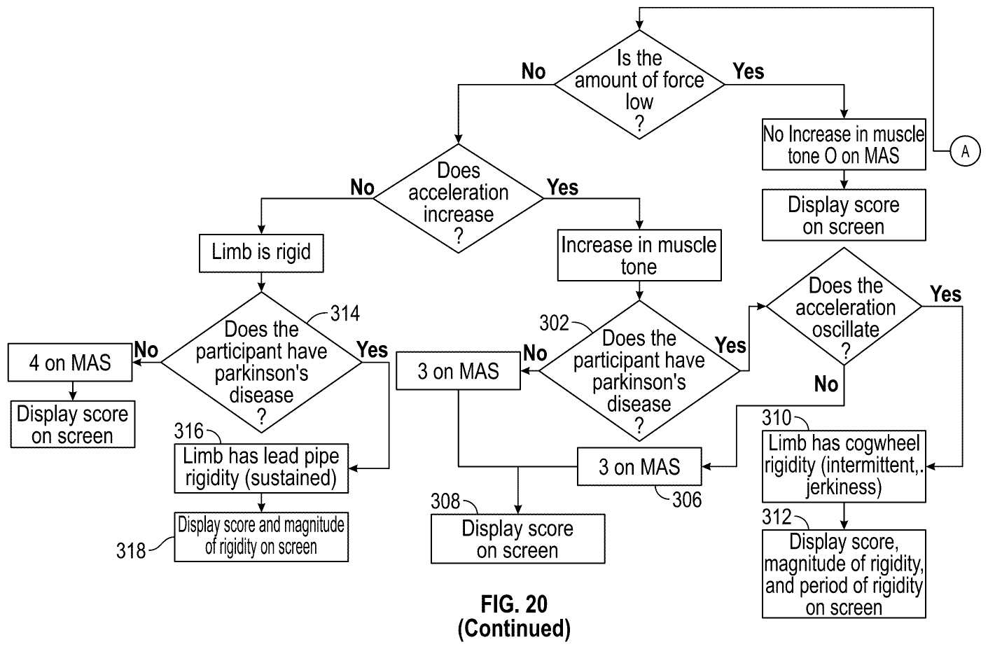

The microcontroller may execute an algorithm 200 for correlating joint response based on data from the IMU and force sensors to one or more clinical measurements (AS, MAS, Tardieu, UPDRS scale of rigidity in Parkinson's Disease). The algorithm, an example of which is illustrated in FIG. 20, may include the following steps and/or it may prompt the user to perform them:

Start: Collect angle, acceleration, and force from a “slow trial” (202). Assessor (e.g., clinician) dons the grasping device on their hand, they grasp the limb of the patient and press the ROM “slow trial” icon on the user interface of the system. The user then slowly (less than 10°/second) rotates the patient's limb through the entirety of the ROM, in both the extension and flexion directions. During this trial, the IMU collects time-stamped angular acceleration, angular velocity, and geomagnetic directions during the entire ROM. Using the geomagnetic sensor, the processor can determine the direction of gravity and obtain the absolute angles of rotation during the trial. The force sensors collect continuous force data, which is passed to the onboard microcontroller to time match with the angular position, velocity, and acceleration data from the IMU.

For each of the “slow” and “fast” trials, the microprocessor may compare the velocity to a set threshold or determine whether the velocity is within a set range based on whether the user is performing the “fast” or “slow” trial. As spasticity is velocity-dependent, it is important to ensure that the user is moving the patient's limb at an appropriate velocity for the particular trial. The system may be configured to notify the user, either via a message on the display or with an auditory alert that the velocity is above or below the set threshold or not within the appropriate range. For example, in the “slow trial”, if the angular velocity exceeds a certain threshold, for example 10°/second, the user will be notified via the screen or a tone.

Step 2: Determine ROM from minimum to maximum angle (204). Once the data starts collecting, the user may delay motion onset. Therefore, to determine the start and end of the motion, the microprocessor will look for a change in position above a preset minimum. This will indicate the start of the motion and that position will be saved as the starting angle and that time will be saved as the starting index. The microprocessor then compares the next angle to the previous angle to determine when the maximum angle occurs. If the next angle is larger than the previous, that angle now becomes the maximum angle. This repeats until the end of the collection. Similar to finding the starting angle, the microprocessor will look for minimum change in position over a small, preset, period of time. If there are no sudden changes in this period, that would indicate the end of the collection and that point will be saved as the ending index.

Step 3: Create force/angle polynomial regression (206). After the data collection is finished, the microprocessor recalls the saved data arrays and fits a polynomial curve to the data points. This may be achieved using a curve-fitting library, for example by Arduino™, which uses a predetermined power and order along with the force and angle vectors to find the coefficients of the polynomial regression.

Step 4: Collect angular position, acceleration, velocity and force data from a “fast trial” (208). The same process occurs as it does for the “slow trial”, but this time the user quickly (e.g. 150°/second) rotates the limb through the entirety of the ROM. For each of the slow and fast trials, the microprocessor may compare the velocity to a set threshold or determine whether the velocity is within a set range based on whether the user is performing the “fast” or “slow” trial. As spasticity is velocity-dependent, it is important to ensure that the user is moving the patient's limb at an appropriate velocity for the particular trial. The system may be configured to notify the user, either via a message on the display or with an auditory alert that the velocity is above or below the set threshold or not within the appropriate range.

Step 5: Analyze acceleration data (210). Once collection is complete, the microprocessor analyzes the acceleration data between the starting and ending indexes of the motion that were saved from the section Determine ROM from starting and maximum angle. A fast Fourier transform (FFT) is performed to compute the discrete Fourier transform of a sequence to determine if there is a sinusoidal response above a threshold.

Step 6: Is there a sinusoidal response? (212) If NO—The frequency did not reach above the specified threshold and returns response=0. (214) The microprocessor tells the display screen this information and the screen displays “No Clonus Present”. (216) If YES—The frequency did reach the specified threshold and returns response=1. (218) The microprocessor tells the display this information and the screen displays “Clonus Present”. (220)

Step 7: Compare the “fast trial” force/angle relationship to the “slow trial” force/angle relationship. (222) The microprocessor retrieves the force data from the “slow trial” and “fast trial”. The “slow trial” force/angle polynomial is then subtracted from the “fast trial” force/angle polynomial between the start and end indexes of the motion to quantify the change in the force/angle relationship.

Step 8: Is there an abrupt change in force? (224) The microprocessor assesses the difference between the fast trial force/angle curve and the slow trial force/angle curve. If the difference reaches a threshold (i.e., the change is abrupt), then a catch is registered and returns catch=1, the index is also recorded at the catch point. Thus, if YES—This means that a catch did occur (catch=1). (226) If the difference does not exceed the threshold, then a catch value=0 is returned. Thus, if NO—This means that no catch occurred during the ROM (catch=0). (228) If the force does not return below that threshold, a release value of 0 is returned, indicating no catch, otherwise a release value of 1 is returned.

Step 9: Find location of abrupt change in force to determine what percentage of ROM the catch happened. (230) The microprocessor identifies the angular position of the catch as the percent of ROM at which the abrupt change in force occurred. To determine the catch percentage, the microprocessor performs the following equation:

$$Catch \% = ( ( catch index - starting index ) / ( ending index - starting index ) ) * 100$$

Step 10: Does the force stay increased after the abrupt change? (232) At this step, the microprocessor assesses force data from the fast trial after a catch has occurred. Is the applied force following the catch significantly more than the slow trial at this joint angle? If NO-This means a release has occurred (release=1) and correlates to a 1 on the Modified Ashworth Scale (MAS). (234) The microprocessor tells the user interface this information and the screen displays “MAS: 1, Catch %: ______”. (236) If YES—This means a release did not occur (release=0) and correlates to a 1+ on the MAS. (238) The microprocessor tells the user interface this information and the screen displays “MAS: 1+, Catch %: ______”. (236)

Step 11: Is the amount of force low? (240) While evaluating the force data, if output values indicate a low amount of force is being applied to the limb, the microprocessor compares the force array to normative data to determine if the force being applied is within the normal range. If NO—This means that more than an average amount of force is being applied. (242) If YES—This means that the limb is easy to move. (244) There is no increase in muscle tone, which correlates to a 0 on MAS. (246) The microprocessor tells the user interface this information and the screen displays “MAS: 0”. (248)

In another embodiment shown in FIG. 20, algorithm 300 may also consider at this step whether the patient has Parkinson's Disease (302). A checkbox may be provided on the main screen of the user interface to indicate this. When checked, the screen tells the microprocessor that PD=1, else it tells that PD=0. If the patient does have Parkinson's Disease (PD=1), the algorithm may include a step of determining whether the acceleration oscillates (304). If NO—This indicates that response=0 (obtained from Is there a sinusoidal response? section) and correlates to a 3 on MAS (306). The microprocessor tells the user interface this information and the screen displays “MAS: 3” (308). If YES—This indicates that that response=1 and the limb has cogwheel rigidity (intermittent, jerkiness) (310). It also correlates to a 3 on MAS. The microprocessor tells the screen this information and the screen displays “MAS: 3” as well as the magnitude of rigidity and period of rigidity (312). The period is the indexes when the oscillation occurs, and the magnitude is determined using the acceleration vector and the following equation:

$$Mag = sqrt ( x \hat{} 2 + y \hat{} 2 + z \hat{} 2 ) .$$

Step 12: Does acceleration increase? (250) The microprocessor retrieves the acceleration data from the IMU for the “fast trial” and determines if there is an increase in acceleration by looking at the acceleration point and the one prior to it. If the difference is above a threshold, it means that the acceleration increases and returns increase=1. Otherwise, there is no increase and returns increase=0.

If NO (252)—There is no increase in acceleration (increase=0) indicating that the limb is rigid (254) which correlates to a MAS of 4 (256). The microprocessor tells the screen this information and the screen displays “MAS: 4” (258). If the participant has Parkinson's Disease (314), the algorithm may, in some embodiments, for example method 300, indicate that the limb has a lead pipe rigidity (sustained) (316). The screen will also display the magnitude of rigidity on the screen (318), which is determined by the acceleration vector and the following equation:

$$Mag = sqrt ( {x}^{⋀} 2 + {y}^{⋀} 2 + {z}^{⋀} 2 )$$

If YES (260)—There is an increase in acceleration (increase=1) indicating that there is an increase in muscle tone (262) which correlates to 3 on MAS (264). The microprocessor tells the screen this information and the screen displays “MAS: 3” (266). In addition to the Modified Ashworth Scale scores, method 200 may also be used to generate other clinically significant outcome measures, including, but not limited to, Tardieu Scale scores.

### Initial Validation Study

Materials and Methods: Data collection included n=8 post-stroke subjects with varying levels of upper extremity spasticity. Hypertonus in the paretic (P) and non-paretic (NP) upper extremities were evaluated by a clinician by extending and flexing the subject's elbow with the use of the MITSS device 3 times at both slow and fast speeds. This process was repeated for 3 pairs of “slow” and “fast” trials with a 2-minute rest period between “slow” and “fast” trials in the P limb and after a 3-minute rest period, the same procedure was performed on the NP limb. Range of motion for both P and NP limbs was recorded using goniometry, and angle of catch for the P limb was also measured. MAS and TS scales were used to assign a score for flexors and extensors for each trial. sEMG for the biceps brachii and triceps for each side was recorded for the entire duration of the experiment. Preliminary data analysis utilized MATLAB R2021a to assess IMU sensor data, namely linear acceleration (m/s2) and relative y-position (degrees) as a function of time. For each “slow” and “fast” trial, the data was separated into separate flexions and extensions using relative joint angle. Points of interest in the ROM for each flexion and extension were detected by time-matching the most prominent peak in linear acceleration with the y-position data for each flexion/extension. Data were then compared between “slow” and “fast” trials, P and NP sides, and the clinician's conventional spasticity evaluation.

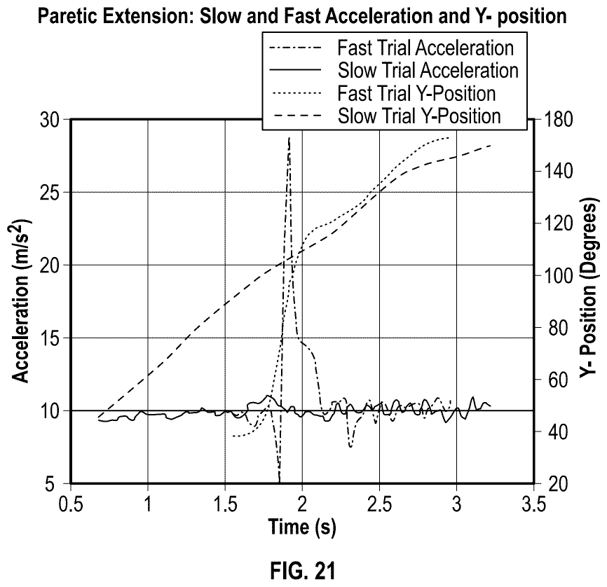

Results and Discussion: A sudden spike in acceleration in a given imposed flexion or extension movement can indicate a sudden stop in the motion, whether it be the end of the motion itself or a possible catch. If the acceleration peaked at 100% ROM, it was assumed to be the stop of the motion. If the acceleration peaked at <100%, this instance was labelled a possible catch occurrence relative to the ROM. Regardless of limb status for “slow trials,” acceleration remained relatively constant with no prominent peaks throughout the ROM as there were no abrupt stops in the passive motion. Therefore, catch occurrence was not detected in either P or NP sides for “slow trials” as expected due to the velocity-dependent characteristic of spasticity. For “fast” NP trials, the acceleration peaked at 100% of the ROM, indicating only the stop of the motion as opposed to a catch, also as expected as there should not have been any catch in the NP side. For the P side, the acceleration generally peaked at <100% of the ROM as shown in FIG. 21, indicating a possible catch occurrence abruptly stopping the motion. IMU data shows peak in acceleration at possible catch occurrence during a “fast trial”; acceleration remains relatively constant for “slow trials” indicating no catch occurrence during slow trials.

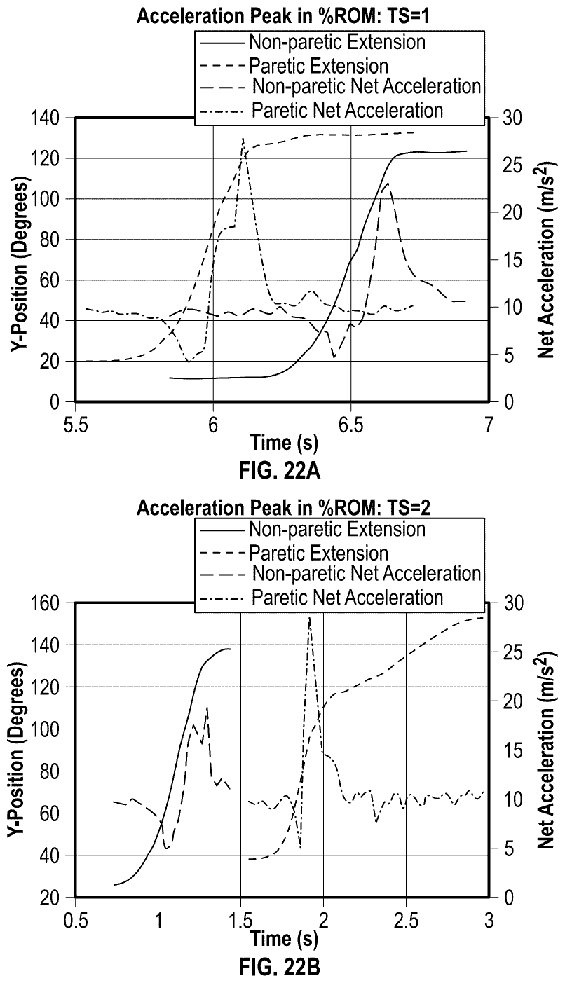

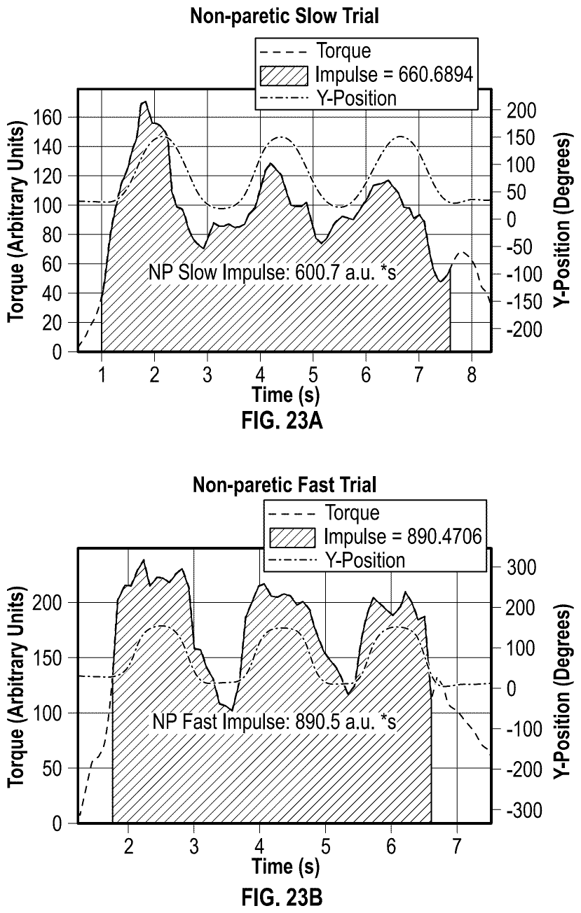

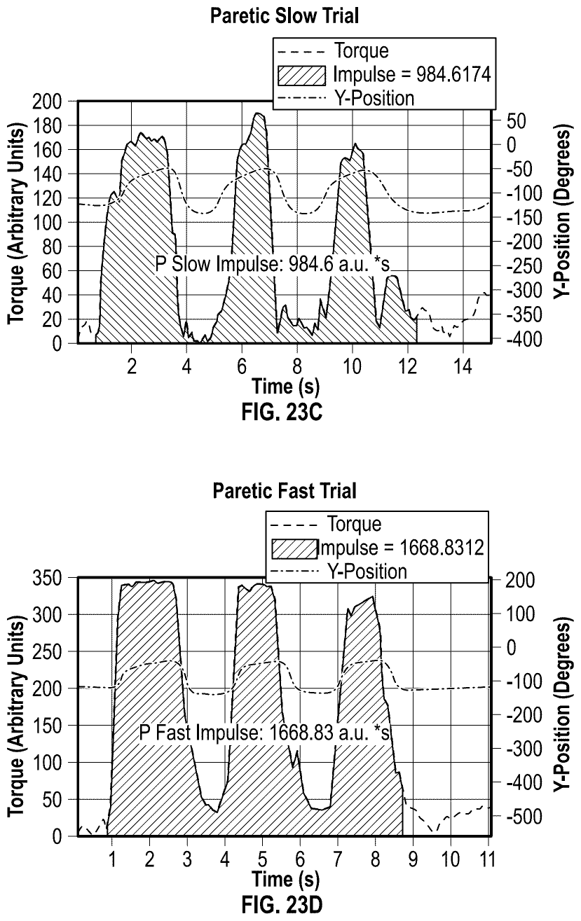

FIGS. 22A and 22B compare the joint angle, acceleration, and angular velocity for the extension of a subject with a TS score of 1 and a TS score of 2, respectively. The negative peak in acceleration occurs as the device approaches a steady state for the linear portion of the joint angle curve and is seen in both paretic and non-paretic limbs. The peak acceleration is concurrent with the change in joint angle. When looking at extension joint angle and acceleration for a trial, there will be a larger peak in acceleration when there is a change in joint angle for the paretic (affected) arm which indicates a “catch”. This is compared to the non-paretic arm, where there is not a significant increase in acceleration or significant change in joint angle. In subjects with less severe spasticity (TS=1), instances of peak net acceleration occurred closer to the end of the ROM (˜100% ROM) following a similar trend as NP “fast trials”. (FIG. 22A). Subjects with more severe scores (TS=2) had instances of peak acceleration occurring much earlier in the ROM which may be due to a muscle catch interrupting the motion. (FIG. 22B). As shown in FIGS. 22A-22D, impulse (area under the torque curve) in paretic trials tended to be greater than in non-paretic trials for both “slow” and “fast” trials, indicating that more force is being applied over time to the paretic side. It is also very likely that subjects with more severe scores have greater impulse differences between NP and P compared to those with less severe scores.

A spasticity model can also be created from a torque-angle curve where the slope represents the stiffness at the joint, and the area within the curve means the energy lost. Information collected with the IMU produces a similar graph, with the angle being identified as the Y-position in degree and the force being represented as the filtered force plate data. A “fast trial” with a higher torque compared to the ROM trial indicates spasticity. For example, in a subject with a MAS score of 3 for the flexor muscles, rotating from flexion to extension for the “fast trial” results in a high torque as compared to the “slow trial”, but a similar torque can be seen going from extension to flexion. This indicates that the subject's flexor muscles are tighter, but little to no spasticity is detected in the extensor muscles.

Although the present invention has been described in certain specific aspects, many additional modifications and variations would be apparent to those skilled in the art. In particular, any of the various processes described above can be performed in alternative sequences and/or in parallel (on different computing devices) in order to achieve similar results in a manner that is more appropriate to the requirements of a specific application. It is therefore to be understood that the present invention can be practiced otherwise than specifically described without departing from the scope and spirit of the present invention. Thus, embodiments of the present invention should be considered in all respects as illustrative and not restrictive.

What is claimed is:

1. A system, comprising:
a device for grasping a limb segment of a patient including a force sensor module configured to generate force data as a limb segment of the patient is moved between a first position and a second position;
at least one inertial measurement unit (IMU) including an accelerometer, magnetometer and gyroscope; and
a processor in operable communication with the force sensor module and the at least one IMU, the processor including instructions that, when executed, cause the processor to:
collect force data from the force sensor module,
collect accelerometer data, gyroscopic data, and geomagnetic data from the at least one IMU, and
generate at least one clinically relevant measure of hypertonus based at least on the force data and one or more of the accelerometer data, gyroscopic data, and geomagnetic data.

2. The system of claim 1, wherein the at least one clinically relevant measure of response is selected from the group consisting of: joint range of motion, presence of catch, percent range of motion at catch, presence of clonus, and score on the Modified Ashworth Scale (MAS).

3. The system of claim 1, wherein the force sensor module includes a first force sensor positioned on a thumb side of the device and a second force sensor positioned on a finger side of the device.

4. The system of claim 3, wherein each of the first force sensor and the second force sensor comprises:
a first plate and a second plate, wherein the first plate is positioned approximately parallel to the second plate;
at least one spring positioned between the first plate and the second plate;
at least one magnet positioned on the first plate; and
at least one magnetic sensor positioned on the second plate,
wherein an output of the magnetic sensor is based, at least in part, on a distance between the first plate and the second plate.

5. The system of claim 4, wherein an output of the magnetic sensor corresponds to a force applied to the first plate and the second plate.

6. The system of claim 4, wherein the instruction to generate at least one clinically relevant measure of hypertonus based at least on the force data and one or more of the accelerometer data, gyroscopic data, and geomagnetic data further comprises:
comparing force data generated by the first force sensor and force data generated by the second force sensor when the limb segment is moved from the first position and the second position and from the second position to the first position.

7. The system of claim 1, comprising a first IMU positioned on the device.

8. The system of claim 7, further comprising a second IMU positioned on a stationary limb segment of the patient.

9. A system, comprising:
a device for grasping the limb of a patient, including a force sensor module for generating force data as a limb segment of the patient is moved between a first position and a second position;
at least one inertial measurement unit (IMU) including an accelerometer, gyroscope, and magnetometer; and
a processor in operable communication with the force sensor module and the at least one IMU, the processor including instructions that, when executed, cause the processor to:
continuously collect force data from the force sensor module as the limb segment of the patient is moved from the first position to the second position for each of a first movement trial and a second movement trial, wherein for the first movement trial the limb segment is moved at a first velocity and wherein for the second movement trial the limb segment is moved at a second velocity;
continuously collect data from the accelerometer, gyroscope, and magnetometer from the at least one IMU as the limb segment of the patient is moved from the first position to the second position for each of the first movement trial and the second movement trial;
for each of the first movement trial and the second movement trial:
generate position data of the limb segment based at least on the collected magnetometer data;
generate acceleration data based at least on the collected accelerometer data;
generate velocity data based at least on the collected gyroscope data; and
generate a correlation of the force data with one or more of the position data, velocity data and acceleration data; and
generate at least one clinically relevant measure of hypertonus based at least on a comparison of the correlation of the force data with one or more of the position data, velocity data and acceleration data for the first movement trial and the correlation of the force data with one or more of the position data, velocity data and acceleration data for the second movement trial.

10. The system of claim 9, wherein the second velocity is greater than the first velocity.

11. The system of claim 9, wherein generating at least one clinically relevant measure of response comprises:
determining a maximum angle of rotation of the limb segment of the patient between the first position and the second position based at least on the position data generated from the first movement trial.

12. The system of claim 9, wherein generating a correlation of the force data with one or more of the position data, velocity data and acceleration data comprises:
generating a force/angle polynomial curve by fitting the force data and the position data to a polynomial curve for each of the first movement trial and the second movement trial.

13. The system of claim 12, wherein generating at least one clinically relevant measure of hypertonus comprises:
identifying the presence of a catch by comparing the force/angle polynomial curve of the first movement trial to the force/angle polynomial curve of the second movement trial and determining if the difference between the force/angle polynomial curve of the first trial and the force/angle polynomial curve of the second trial is above a threshold.

14. The system of claim 13, wherein generating at least one clinically relevant measure of hypertonus further comprises:
identifying an angular position of the catch as the position between the first position and the second position where the difference between the force/angle polynomial curve of the first trial and the force/angle polynomial curve of the second trial is above a threshold.

15. A method, comprising:
providing a device for grasping the limb of a patient, including at least one inertial measurement unit (IMU) and a force sensor module for continuously measuring a force generated by moving the limb of the patient between a first position and a second position;
causing a processor in operable communication with the force sensor module and the at least one IMU, to:
continuously collect force data from the force sensor module as the limb segment of the patient is rotated from the first position to the second position for each of a first movement trial and a second movement trial, wherein for the first movement trial the limb segment is rotated at a first velocity and wherein for the second movement trial the limb segment is rotated at a second velocity;
continuously collect inertial data from the at least one IMU as the limb segment of the patient is rotated from the first position to the second position at the first velocity and the second velocity, wherein the inertial data includes accelerometer data, gyroscope data and geomagnetic data; and
for each of the first movement trial and the second movement trial:
generate position data of the limb segment based at least on the collected magnetometer data;
generate acceleration data based at least on the collected accelerometer data;
generate velocity data based at least on the collected gyroscope data;
correlate the force data with one or more of the position data, velocity data and acceleration data; and
determine a maximum angle of rotation of the limb segment of the patient between the first position and the second position based at least on the position data generated from the first movement trial; and
generate at least one clinically relevant measure of hypertonus based at least on a correlation of the force data with one or more of the position data, velocity data and acceleration data.

16. The method of claim 15, wherein generating at least one clinically relevant measure of hypertonus comprises:
generating a force/angle polynomial curve by fitting the force data and the angular position data to a polynomial curve for each of the first movement trial and the second movement trial; and
identifying the presence of a catch by comparing the force/angle polynomial curve of the first movement trial to the force/angle polynomial curve of the second movement trial.

17. The method of claim 16, wherein the presence of the catch is identified where the difference between the force/angle polynomial curve of the first trial and the force/angle polynomial curve of the second trial is above a threshold.

18. The method of claim 17, wherein generating at least one clinically relevant measure of hypertonus comprises:
identifying an angular position of the catch as the position between the first position and the second position where the difference between the force/angle polynomial curve of the first trial and the force/angle polynomial curve of the second trial is above a threshold.

19. The method of claim 15, wherein generating at least one clinically relevant measure of hypertonus comprises:
generating a score on the Modified Ashworth Scale based on the correlation of the force data with one or more of the position data, velocity data and acceleration data.
